(19) United States
(12) Patent Application Publication
(10) Pub. No.: US 2023/0277168 A1
(43) Pub. Date: Sep. 7, 2023

(54) A HUMAN IMPLANT ARRANGEMENT FOR CONSTRICTING A HUMAN TISSUE STRUCTURE

(71) Applicant: PROMEDON GMBH, KOLBERMOOR (DE)

(72) Inventor: OSVALDO NICOLAS GRIGUOL, CORDOBA (AR)

(73) Assignee: PROMEDON GMBH, KOLBERMOOR (DE)

(21) Appl. No.: 18/018,043

(22) PCT Filed: Jul. 29, 2020
(86) PCT No.: PCT/EP2020/071429

§ 371 Date: Jan. 26, 2023

(51) Int. Cl. A61B 17/04

(52) U.S. Cl. CPC A61B 17/0401; A61B 2017/0409; A61B 2017/0414; A61B 2017/0446; A61F 2250/006

(57) ABSTRACT

A human implant arrangement for permanently constricting a human tissue structure in a longitudinal direction. The human implant arrangement includes a longitudinal first anchoring part having lateral self-anchoring elements which have a first longitudinal anchoring orientation, a connection part which does not have anchoring elements, and a longitudinal second anchoring part which is connected in a longitudinal orientation to the longitudinal first anchoring part via the connection part. The longitudinal second anchoring part has lateral self-anchoring elements which have a second longitudinal anchoring orientation which is reverse to the first longitudinal anchoring orientation. A total length of the human implant arrangement is not larger than 40 mm.

### CROSS REFERENCE TO PRIOR APPLICATIONS

This application is a U.S. National Phase application under 35 U.S.C. § 371 of International Application No. PCT/EP2020/071429, filed on Jul. 29, 2020. The International Application was published in English on Feb. 3, 2022 as WO 2022/022821 A1 under PCT Article 21(2).

### FIELD

The present invention relates to a human implant arrangement for permanently constricting, plicating or reinforcing a human tissue structure, in particular for constricting a pelvic ligament, and even more particularly for constricting a female pelvic floor ligament or a female urinary ligament.

### BACKGROUND

In the state of the art, sling-like implant arrangements are used to support human urinary tissue structures as a therapy of urinary incontinence. WO 2005/107606 A1, WO 2003/086205 A2 and EP 2 170 218 B1 describe human urinary implant arrangements which are designed and implanted as sling-formed structures for mechanically supporting the urethra. The implant arrangements are provided with a relatively large central middle-section for supporting the urinary tissue structure or a uterus structure so that the total length of the implant arrangements is between 40 mm and 150 mm. The sling middle section is held by lateral anchoring parts which are anchored in a human tissue structure at the left and at the right human body side.

### SUMMARY

An aspect of the present invention is to provide an alternative human implant arrangement for therapy of weak human tissue.

In an embodiment, the present invention provides a human implant arrangement for permanently constricting a human tissue structure in a longitudinal direction. The human implant arrangement includes a longitudinal first anchoring part comprising lateral self-anchoring elements having a first longitudinal anchoring orientation, a connection part which does not comprise anchoring elements, and a longitudinal second anchoring part which is connected in a longitudinal orientation to the longitudinal first anchoring part via the connection part. The longitudinal second anchoring part comprises lateral self-anchoring elements having a second longitudinal anchoring orientation which is reverse to the first longitudinal anchoring orientation. A total length of the human implant arrangement is not larger than 40 mm.

### BRIEF DESCRIPTION OF THE DRAWINGS

The present invention is described in greater detail below on the basis of embodiments and of the drawings in which:

FIG. 1 shows a first embodiment of the human implant arrangement defined by a single integral plastic body;

FIG. 2 also shows the first embodiment of the human implant arrangement defined by the single integral plastic body;

FIG. 3 shows the human implant arrangement of FIGS. 1 and 2 provided with an application sleeve;

FIG. 4 shows an application tool engagement structure of the human implant arrangement of FIGS. 1 to 3 including an application tool end section;

FIG. 5 shows the application tool, the implant arrangement, and the application sleeve of FIGS. 1 to 4;

FIG. 6 shows a human urinary tissue site with two implanted human implant arrangements;

FIG. 7 shows a second embodiment of the human implant arrangement with a first embodiment of a length adjusting device;

FIG. 8 also shows the second embodiment of the human implant arrangement with a first embodiment of a length adjusting device;

FIG. 9 shows a third embodiment of the human implant arrangement with a second variation of a length adjusting device;

FIG. 10 shows a fourth embodiment of the human implant arrangement with a third variation of a length adjusting device;

FIG. 11 shows a human pelvic tissue site with two implanted human implant arrangements of the fourth embodiment;

FIG. 12 shows a fifth embodiment of the human implant arrangement with self-anchoring elements lying in several longitudinal planes at the second anchoring part;

FIG. 13 shows a sixth embodiment of the human implant arrangement with a modular concept of the second anchoring part; and

FIG. 14 also shows the sixth embodiment of the human implant arrangement with a modular concept of the second anchoring part.

### DETAILED DESCRIPTION

According to the present invention, the human implant arrangement is provided with a longitudinal first anchoring part with lateral inclined self-anchoring elements with a first longitudinal anchoring direction, an adjacent connection part without any anchoring element and, adjacent to the connection part, a longitudinal second anchoring part which is connected in longitudinal orientation to the first anchoring part by the interjacent connection part. The second anchoring part is provided with inclined lateral self-anchoring elements with a second longitudinal anchoring orientation being reverse to the first anchoring orientation of the first anchoring part. Both anchoring orientations are directed proximal to the middle, i.e., to the connection part between the two anchoring parts.

The total length of the implant arrangement is not larger than 40 mm. Since the implant arrangements longitudinal extension is relatively short, the implant arrangement is not suitable to be used as a sling and is not suitable to directly support a human tissue structure, e.g., the urethra, with a middle section of the implant arrangement. The claimed human implant arrangement is suitable and is used to be implanted and fixed directly at a pelvic ligament, e.g., at a female pubourethral ligament, a pubourethral ligament, or the uterosacral ligament, or to restore and tighten another human tissue structure. In the final implanted position of the implant arrangement, the free ends of the self-anchoring elements of the two counter-acting anchoring parts face each other so that the implant arrangement thereby permanently pulls together the human tissue structure in the longitudinal direction of the implant arrangement. The effective length of the human urinary tissue structure can thereby be stabilized and/or shortened.

The self-anchoring elements can, for example, be lateral spine bodies which are ending acute and pointed at their free ends so that the self-anchoring elements easily pull themselves into a human urinary tissue structure if they are pulled in the anchoring direction.

The implanting process starts with introducing the implant arrangement with the first anchoring part ahead, and the second anchoring part following. When the first anchoring part has arrived at the final implantation location, the implant arrangement can be pulled somewhat to force the self-anchoring elements of the first anchoring part to anchor in the surrounding human urinary tissue structure. The human urinary tissue structure downstream of the first anchoring part can then be pushed upstream until somewhat more than the desired total shortening of the human tissue structure is achieved. Finally, any external force is released so that the treated human tissue structure relaxes and the self-anchoring elements of the second anchoring part finally anchor in the surrounding human urinary tissue structure. The human urinary tissue structure around the complete longitudinal length of the implanted implant arrangement is thereby permanently shortened and/or stabilized in the longitudinal direction.

According to an additional or alternative aspect of the present invention, the lateral extension of the connection part is not larger than the lateral extension of the two anchoring parts. Since the connection part between the two anchoring parts has no supporting function, but must simply hold together the two anchoring parts and keep a distance between the anchoring elements, the connection part need not have a substantive support surface size. The connection part can, for example, be relatively small so that the human tissue structure is not radially expanded in this region. Since the tissue structure is not expanded in this region, the self-anchoring elements located closely to the connection part remain perfectly seated in the human urinary tissue structure.

In an embodiment of the present invention, the connection part can, for example, be provided with a ring structure, where the ring plane of the ring structure lies in the plane of the self-anchoring elements of both anchoring parts. In other words, all self-anchoring elements of both anchoring parts as well as the connection part ring structure substantially lie in one single longitudinal plane. The corresponding human urinary tissue structure being supported by the implanted implant arrangement thereby also remains relatively compact and is only expanded in one longitudinal plane and therefore remains relatively compact.

The first anchoring part, the connection part, and the second anchoring part can, for example, be defined by one single integral plastic body. The implant arrangement is made of a biocompatible material, is sufficiently elastic and, even as a filigree structure, is strong and steady enough for serving as an implant in a human urinary tissue structure.

In an embodiment of the present invention, the longitudinal length of the connection part can, for example, be less than the longitudinal length of any one of the two self-anchoring parts. The longitudinal length of the connection part can, for example, not be higher than ¼ of the total longitudinal length of the complete implant arrangement. Since the connection part only has the function to connect the two counter-acting self-anchoring parts, the connection part without any anchoring elements can be relatively short. The available longitudinal length for providing self-anchoring elements is thereby optimized so as to improve the self-anchoring quality.

The included anchor angle of all self-anchoring elements can, for example, be less than 55°. This means that the included anchor angle of the self-anchoring elements is relatively small so that a reliable self-anchoring effect is provided even if the respective human urinary tissue structure should intensively be moved by surrounding muscle activity.

In an embodiment of the present invention, the total lateral extension of the implant arrangement in the longitudinal plane of the anchoring parts can, for example, not be larger than 10 mm. The total lateral extension can, for example, be less than 300 % of the lateral thickness of the center body holding the self-anchoring elements. In other words, the center body holding the self-anchoring elements is as slim as possible to allow a maximum lateral extension and maximum total length of the self-anchoring elements.

Only the second anchoring part can, for example, be provided with self-anchoring elements lying in at least two different anchoring element planes.

In an embodiment or an alternative embodiment of the present invention, at least the secondary anchoring part can, for example, be provided with and covered by a removable application sleeve which laterally covers every self-anchoring element of the second anchoring part. The self-anchoring elements of the second anchoring part are not effective as long as the application sleeve is present. The removable application sleeve can additionally cover the connection part and the first anchoring part, however, this is not absolutely necessary. The application sleeve makes a pushing of the second anchoring part through the respective human urinary tissue structure against the anchoring orientation of the second anchoring part possible. Since the present implant arrangement is not implanted to define a sling, but is supposed to be implanted as a tissue pulling element, both anchoring parts are introduced into the same respective human tissue structure in one single direction so that the second anchoring part is introduced against its anchoring orientation. The implantation of the implant arrangement is substantially simplified by using the application sleeve. When the implant arrangement has arrived at the correct location within the human urinary tissue structure, the application sleeve is removed so that the self-anchoring elements of the second anchoring part are undressed and become effective as self-anchoring elements, and can anchor in the surrounding human tissue structure. This aspect of the present invention is applicable even for implant arrangements which are much longer than 40 mm.

In an embodiment of the application sleeve of the present invention, a releasable sleeve retainer element is provided to hold the application sleeve in place at one of the three parts of the implant arrangement as long as the sleeve retainer element is not released. The sleeve retainer element is provided with a release device for easily releasing the sleeve retainer element. The sleeve retainer element can, for example, be a thin thread, a wire, a filament etc. which can be pulled remotely to release the sleeve retainer element so that the application sleeve can be released and removed after the implant arrangement has been set in place in the human urinary tissue structure.

In an embodiment of the application sleeve of the present invention, the application sleeve can, for example, be provided to be removable in longitudinal direction after being released. Since the implant arrangement is implanted via a movement in a longitudinal direction, the application sleeve can simply be removed from the respective tissue structure in the reverse implantation direction of the implant arrangement.

Only the free longitudinal end of the first anchoring part which is the ahead part during the implantation movement can, for example, be provided with an application tool engagement structure for engagement of a releasable application tool. The free end of the second anchoring part is not provided with any tool engagement structure because the implant arrangement is completely implanted by one single implantation movement in one single direction or one single orientation. The application tool can be provided as a handle with a stiff guide sleeve for pulling the implant arrangement at the ahead free end of the first anchoring part.

In an alternative embodiment of the present invention, the connection part between the two anchoring parts can, for example, be provided with a length adjusting device for adjusting the longitudinal distance between the two anchoring parts. The length adjusting device makes it is possible to adjust the longitudinal distance between the anchoring parts and to adjust the total length of the implant arrangement. The length adjusting device can be operated during the implantation action so that the implant arrangement can be adapted to the tissue structure during the implantation action. The length adjusting device can generally be provided with different length adjusting techniques and elements, and is also applicable for implant arrangements exceeding 40 mm length.

The length adjusting device can, for example, be provided with a unidirectional snap-in structure which only allows a shortening of the longitudinal length of the implant arrangement, but which does not allow the total length of the implant arrangement to be increased. The length adjusting device is easily operable during the implantation action and is safe against an unintentional elongation of the implant arrangement after being implanted into the human urinary tissue structure.

In an alternative embodiment of the present invention, the second anchoring part can, for example, be provided with a holder element which holds at least one separate anchoring module in place. The second anchoring part therefore has a modular concept and allows the size and the structure of the self-anchoring elements of the second anchoring part to be individually adapted. The self-anchoring elements can have different lengths, different angles, different orientations, different strengths etc. The holder element can, for example, be a holding stick to which one or more anchoring modules can be attached by plugging a corresponding bore of the anchoring module onto the holding stick. This modular concept is generally also applicable for all types and sizes of implant arrangements being provided with an anchoring part.

Several embodiments of the present invention are described below with reference to the enclosed drawings.

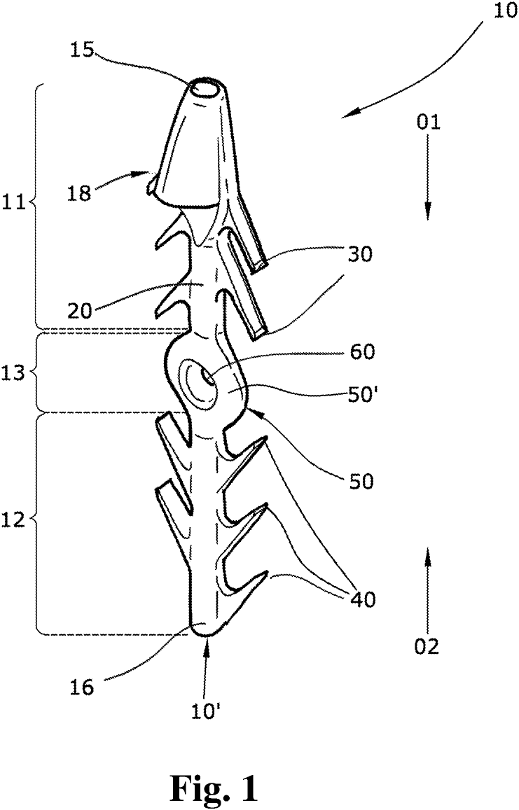

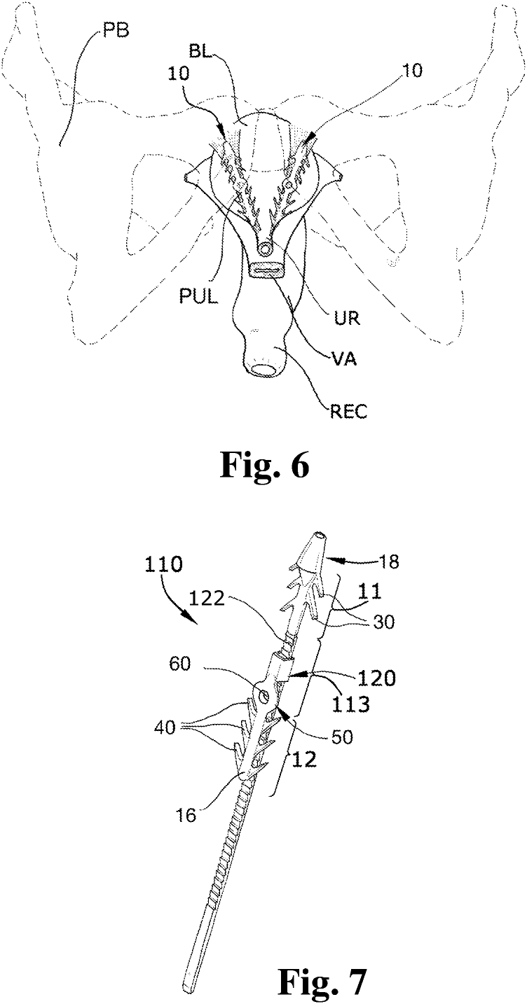

FIGS. 1 to 6 show a first embodiment of a human implant arrangement 10 being made of a single integral plastic body 10′ of a biocompatible plastic material, for example, a biocompatible polymer. In the described embodiments, the implant arrangement 10 is suitable and used for permanently constricting or placating a human urinary tissue structure PUL which is, in the medical application shown in FIG. 6, each of the two pubourethral ligaments PUL of the female human body.

The implant arrangement 10 according to the first embodiment is provided with substantially three functional parts, namely, a longitudinal first anchoring part 11, a longitudinal second anchoring part 12, and a connection part 13 connecting the first anchoring part 11 and the second anchoring part 12 in longitudinal direction along the longitudinal axis A of the single integral plastic body 10′. The total length L of the implant arrangement 10 is about 20 mm, and the total lateral extension W is about 5 mm. The thickness of the implant arrangement 10 is less than 1.2 mm. The single integral plastic body 10′ of the implant arrangement 10 has a first longitudinal end 15 which is the proximal end, and has a second longitudinal end 16 which is the distal end.

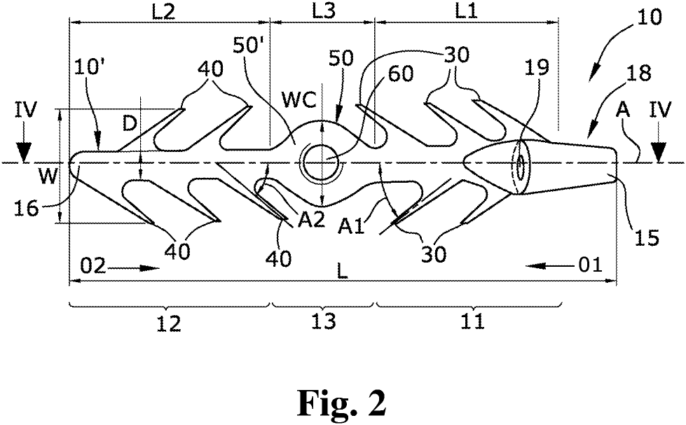

The longitudinal first anchoring part 11 is provided with a total of five lateral self-anchoring elements 30 with a first longitudinal anchoring orientation O1, whereas the longitudinal second anchoring part 12 is provided with five lateral self-anchoring elements 40 with a second longitudinal anchoring orientation O2 which is reverse/opposite to the first longitudinal anchoring orientation O1. The self-anchoring elements 30, 40 of both anchoring parts 11, 12 are designed as tapered spines or needles which define an included anchor angle A1, A2 of about 40° as shown in FIG. 2.

The connection part 13 is provided with a first ring structure 50 which is defined by a ring body 50′ around an ring structure opening 60. The ring plane of the first ring structure 50 and the general plane of the self-anchoring elements 30, 40 of both anchoring parts 11, 12 lie within one single longitudinal plane. The longitudinal length L3 of the connection part 13 as defined by the first ring structure 50 is in the present embodiment less than 4 mm which is less than ¼ of the total longitudinal length L of the complete implant arrangement 10. The longitudinal length L1, L2 of each of the self-anchoring parts 11, 12 is between 5 and 8 mm so that the longitudinal length each of the self-anchoring parts 11, 12 is substantially higher than the longitudinal length of L3 of the connection part 13. Most of the total length L of the human implant arrangement 10 is used for the self-anchoring parts 11, 12.

The total lateral extension W of the implant arrangement 10 in the plane of the anchoring parts is 5 mm, whereas the lateral thickness D of a center body 20 holding the self-anchoring elements 30, 40 of the first anchoring part 11 and the second anchoring part 12 is less than 1.5 mm, so that the total lateral extension W is at least three times higher than the lateral thickness D of the of the center body 20. The lateral extension WC of the connecting part 12 is about 4 mm, and is therefore smaller than the lateral extension W of the anchoring parts 11, 12, but is larger than the lateral thickness D of the center body 20.

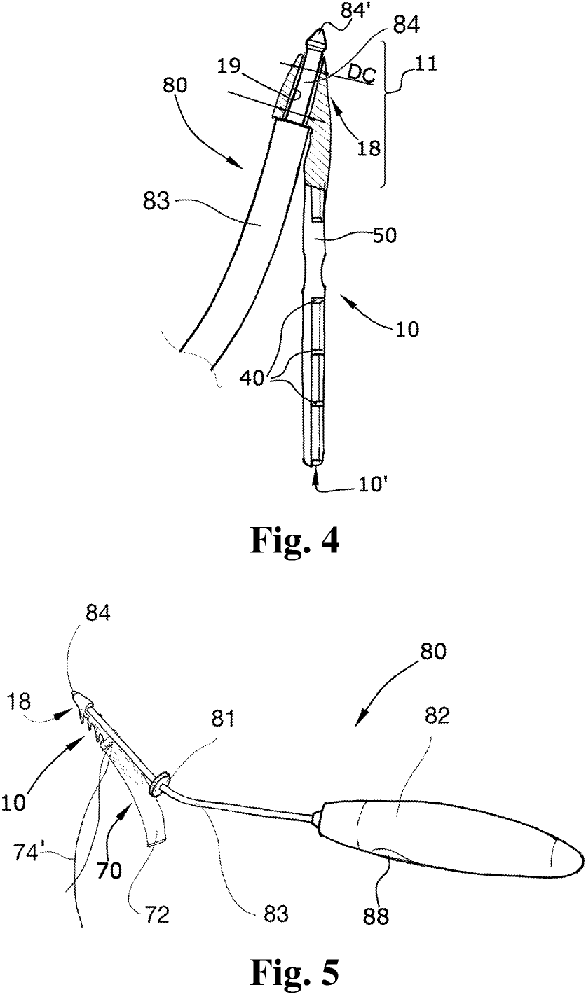

The proximal free first longitudinal end 15 of the implant arrangement 10 is provided with an application tool engagement structure 18 for engagement of a releasable application tool 80 which is shown in FIGS. 4 and 5. The application tool engagement structure 18 is basically defined by a cylindrical engagement bore 19 which is inclined in an angle of about 10° with respect to the longitudinal plane of the self-anchoring elements 30, 40.

The releasable application tool 80 is provided with a handle 82 and a stiff hollow Bowden cable guiding sleeve 83 enclosing and guiding a release cable 84 with a release cable head 84′ at the proximal end of the release cable 84. The cable guiding sleeve 83 is provided with an outside stopping ring 81 in a distance of about 30 mm from the release cable head 84′. The outside diameter DC of the release cable 84 is smaller than the inner diameter of the engagement bore 19, whereas the outside diameter of the release cable head 84′ is at least minimally larger than the inner diameter of the engagement bore 19.

The implant arrangement 10 is introduced and positioned in the human urinary tissue structure by using the releasable application tool 80 as shown in FIG. 6. As soon as the implant arrangement 10 is in place, a release button 88 at the handle 82 is pushed so that the release cable head 84′ is completely pulled through the engagement bore 19 in the distal direction so that the releasable application tool 80 can finally be completely withdrawn from the implant arrangement 10 and the urinary tissue structure.

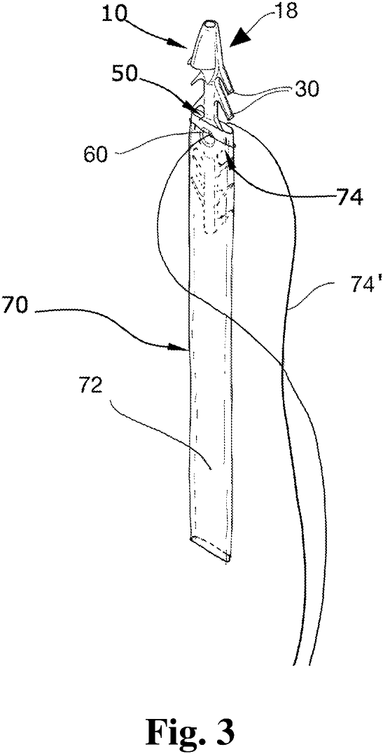

FIG. 3 shows the implant arrangement 10 with an applied application sleeve 70 which laterally completely covers the second anchoring part 12 including all self-anchoring elements 40 of the second anchoring part 12. The application sleeve 70 is defined by a tubelike flexible sleeve foil 72 of a biocompatible plastic material and protrudes distally from the second anchoring part 12 in the longitudinal direction by at least 20 mm. The application sleeve 70 is longitudinally removable when released so that the application sleeve 70 can be pulled away from the implant arrangement 10 in the longitudinal distal direction.

The application sleeve 70 is releasably held by a releasable sleeve retainer element 74 at the connection part 13 during the medical application process. The releasable sleeve retainer element 74 is, in this embodiment, a release thread 74′ which protrudes through the ring structure opening 60 of the first ring structure 50 of the connection part 13. After the implant arrangement 10 has been positioned correctly in its final position in the human body, the release thread 74′ is pulled at one of the two free ends of the release thread 74′, so that a closed loop of the release thread 74′ at the ring structure opening 60 is opened or the release thread 74′ is ripped into pieces so that the application sleeve 70 can be removed in a longitudinal distal direction from the implant arrangement 10. The release thread 74′ is alternatively pulled at one end, and consequently slides through the ring structure opening 60 until the opposite thread end has passed the ring structure opening 60.

FIG. 6 shows a front view of the human female urinary site with the pubic bone PB, the vagina VA, the rectum REC, the bladder BL, the urethra UR, and two pubourethral ligaments PUL. Two separate implant arrangements 10 are implanted to be positioned parallel and alongside each of the pubourethral ligaments PUL.

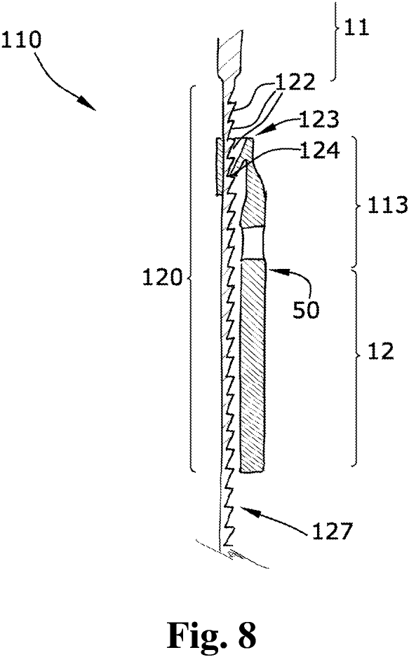

FIGS. 7 and 8 show a second alternative implant arrangement 110 comprising a length adjusting device 120 for adjusting the longitudinal distance between the anchoring parts 11, 12. The length adjusting device 120 is provided with a unidirectional snap-in structure 123 which is basically defined by a toothed rack 127 continuing the first anchoring part 11 longitudinally in the distal direction and being provided with a plurality of non-symmetric sawtooth-like teeth 122 and by a co-acting snap-in tongue 124 of the connection part 113. This snap-in structure 123 is known, for example, from plastic cable ties. In this embodiment of the alternative implant arrangement 110, the longitudinal length of the connection part 113 not being provided with self-anchoring elements can individually be adapted to the urinary tissue structure site during the implantation action. The present snap-in structure 123 can be adapted only unidirectionally so that the longitudinal length of the connection part 113 can only be shortened. The unidirectional characteristics of the length adjusting device 120 provides that the implant arrangement 110 cannot be lengthened after implantation. After the final length of the connection part 113 has been adjusted, the distal part of the toothed rack 127 can be cut off and removed.

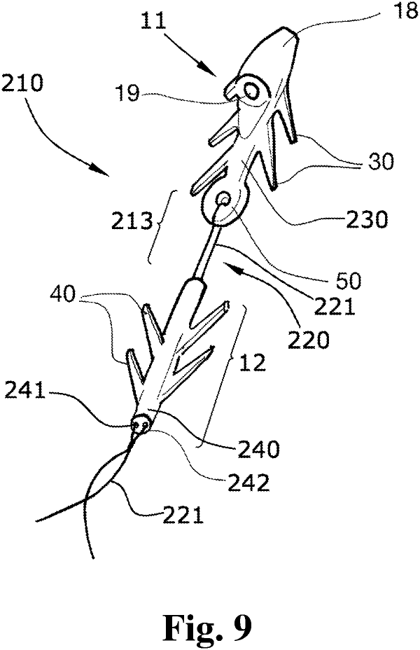

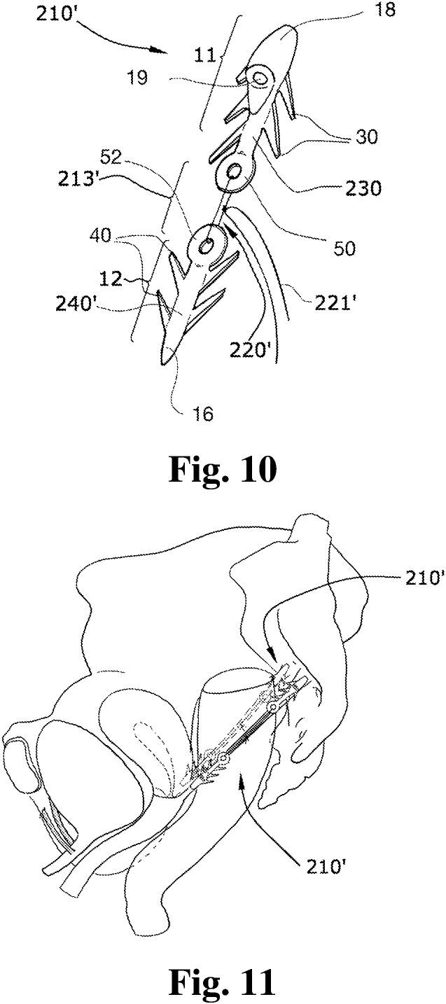

FIGS. 9 and 10 show two other alternative embodiments of human urinary implant arrangements 210, 210′ with connection parts 213, 213′ being provided with adjustable length adjusting devices 220, 220′ realized by an adjusting thread 221, 221′.

FIG. 11 shows a prolapse use of the human implant arrangement 210′ of the fourth embodiment as shown in FIG. 10. The anchoring parts 11, 12 are respectively anchored in each extremity of the lax o torn uterosacral ligaments to plicate them, achieving uterus repositioning to its original position and consequently prolapse treatment.

In the embodiment of the implant arrangement 210 of FIG. 9, the adjusting thread 221 is looped through the first ring structure 50 being provided integral with the body 230 of the first anchoring part 11. Both arms of the open threat loop are led through longitudinal bores 241, 242 in the body 240 of the second anchoring part 12. The longitudinal length of the implant arrangement 210 can be adjusted by shortening or elongating the thread loop between the first ring structure 50 and the second anchoring part 12 and by finally tying and knotting the free ends of the adjusting thread 221.

In the embodiment of the implant arrangement to 210′ of FIG. 10, the adjusting thread 221′ connects with a closed loop the first ring structure 50 being integral with the first anchoring part 11, and with a second ring structure 52 being provided integral with the second anchoring part 12.

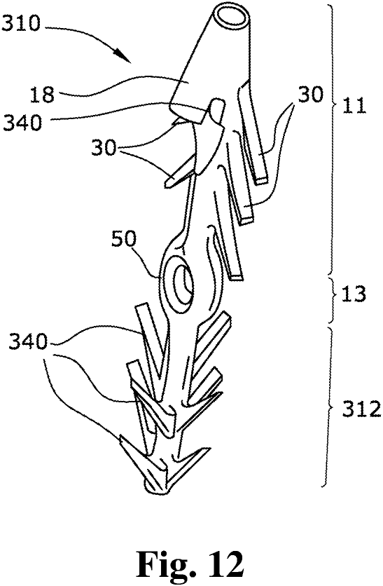

In another alternative embodiment shown in FIG. 12, the implant arrangement 310 is provided with a second anchoring part 312 with self-anchoring elements 340 lying in two different longitudinal planes. The first anchoring part 11 is provided with self-anchoring elements 30 lying in one single longitudinal plane.

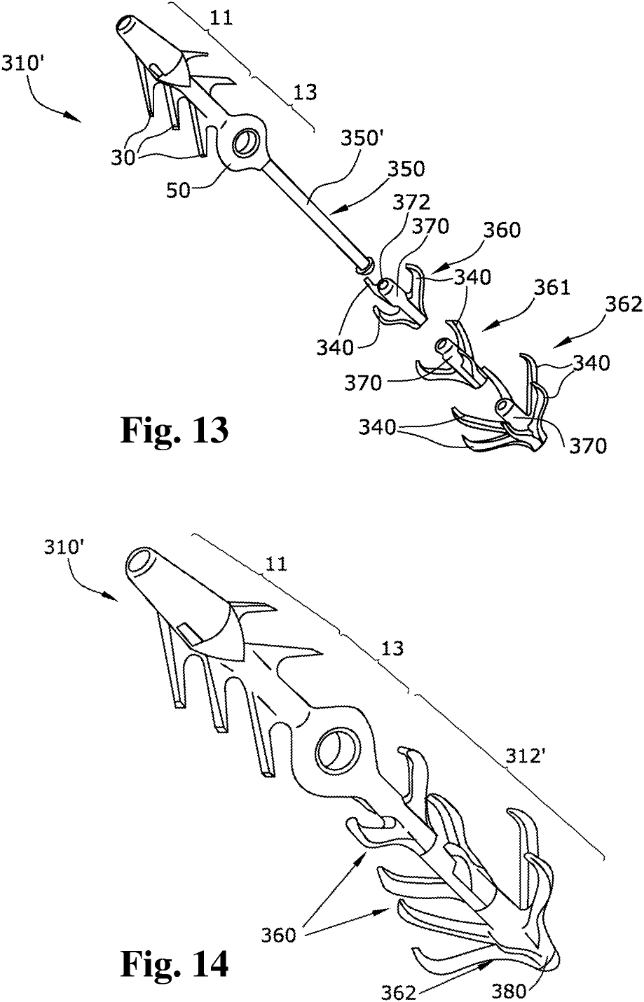

In another alternative embodiment shown in FIGS. 13 and 14, the implant arrangement 310′ is provided with a modular second anchoring part 312′. The second anchoring part 312′ is provided with a holder element 350 which is realized as a holding stick 350′ holding three separate anchoring modules 360, 362 of different types. The individual combination of anchoring modules of the second anchoring part 312′ is chosen and assembled before the implant arrangement 310′ is implanted.

Every anchoring module 360, 362 comprises a hollow sleeve body 370 with a longitudinal sleeve bore 372, whereas the self-anchoring elements 340 are integrally connected to the corresponding hollow sleeve body 370. The individual second anchoring part 312′ can be created by subsequently shifting, in this example, three anchoring modules 360, 362 onto the holding stick 350′. The anchoring modules 360, 362 can be provided co-rotatably with the holding stick 350′ so that the anchoring modules 360, 362 do not rotate with respect to the second anchoring part 312′. After the anchoring modules 360, 362 have been placed at the holding stick 350′, the distal end of the holding stick 350′ is provided with a stop knob 380 so that the anchoring modules 360, 362 are longitudinally blocked at the holding stick 350′. The stop knob 380 can be provided by heat treatment and/or by deforming of the distal end of the holding stick 350′ to define a stop knob 380.

The present invention is not limited to embodiments described herein; reference should be had to the appended claims.

LIST OF REFERENCE CHARACTERS

| | |
|---|---|
| 01 | First longitudinal anchoring orientation |
| 02 | Second longitudinal anchoring orientation |
| 10 | Implant arrangement |
| 10′ | Single integral plastic body |
| 11 | First anchoring part |
| 12 | Second anchoring part |
| 13 | Connection part |
| 15 | First longitudinal end |
| 16 | Second longitudinal end |
| 18 | Application tool engagement structure |
| 19 | Engagement bore |
| 20 | Center body |
| 30 | Self-anchoring element |
| 40 | Self-anchoring element |
| 50 | First ring structure |
| 50′ | Ring body |
| 52 | Second ring structure |
| 60 | Ring structure opening |
| 70 | Application sleeve |
| 72 | Sleeve foil |
| 74 | Releasable sleeve retainer element |
| 74′ | Release thread |
| 80 | Releasable application tool |
| 81 | Outside stopping ring |
| 82 | Handle |
| 83 | Cable guiding sleeve |
| 84 | Release cable |
| 84′ | Release cable head |
| 88 | Release button |
| 110 | Implant arrangement |
| 113 | Connection part |
| 120 | Length adjusting device |
| 122 | Sawtooth-like tooth |
| 123 | Unidirectional snap-in structure |
| 124 | Snap-in tongue |
| 127 | Toothed rack |
| 210 | Implant arrangement |
| 210′ | Implant arrangement |
| 213 | Connection part |
| 213′ | Connection part |
| 220 | Length adjusting device |
| 220′ | Length adjusting device |
| 221 | Adjusting thread |
| 221′ | Adjusting thread |
| 230 | Body (of first anchoring part 11) |
| 240 | Body (of second anchoring part 12) |
| 240′ | Body (of second anchoring part 12) |
| 241 | Longitudinal bore |
| 242 | Longitudinal bore |
| 310 | Implant arrangement |
| 310′ | Implant arrangement |
| 312 | Second anchoring part |
| 312′ | Second anchoring part |
| 340 | Self-anchoring element |
| 350 | Holder element |
| 350′ | Holding stick |
| 360 | Anchoring module |
| 361 | Anchoring module |
| 362 | Anchoring module |
| 370 | Hollow sleeve body |
| 372 | Sleeve bore |
| 380 | Stop knob |
| A | Longitudinal axis |
| A1 | Anchor angle |
| A2 | Anchor angle |
| BL | Bladder |
| D | Lateral thickness (of center body 20) |
| DC | Outside diameter (of release cable 84) |
| L | Total length (of complete implant arrangement 10) |
| L1 | Longitudinal length (of self-anchoring part 11) |
| L2 | Longitudinal length (of self-anchoring part 12) |
| L3 | Longitudinal length (of connection part 13) |
| PB | Pubic bone |
| REC | Rectum |
| PUL | Urinary tissue structure/Pubourethral ligaments |
| UR | Urethra |
| VA | Vagina |
| W | Total lateral extension |
| WC | Lateral extension |

What is claimed is:

1-15. (canceled)

16. A human implant arrangement for permanently constricting a human tissue structure in a longitudinal direction, the human implant arrangement comprising:
a longitudinal first anchoring part comprising lateral self-anchoring elements having a first longitudinal anchoring orientation;
a connection part which does not comprise anchoring elements; and
a longitudinal second anchoring part which is connected in a longitudinal orientation to the longitudinal first anchoring part via the connection part, the longitudinal second anchoring part comprising lateral self-anchoring elements having a second longitudinal anchoring orientation which is reverse to the first longitudinal anchoring orientation, wherein,
a total length of the human implant arrangement is not larger than 40 mm.

17. The human implant arrangement as recited in claim 16, wherein the longitudinal second anchoring part further comprises a removable application sleeve which laterally covers each of the lateral self-anchoring elements of the longitudinal second anchoring part.

18. The human implant arrangement as recited in claim 17, further comprising:
a sleeve retainer element, wherein,
the removable application sleeve is releasably held by the sleeve retainer element at one of the longitudinal first anchoring part, the longitudinal second anchoring part, and the connection part.

19. The human implant arrangement as recited in claim 17, wherein the removable application sleeve is configured to be removable in a longitudinal direction when released.

20. The human implant arrangement as recited in claim 16, further comprising:
a releasable application tool, wherein,
the longitudinal first anchoring part further comprises a free longitudinal end which comprises an application tool engagement structure which is configured to engage with the releasable application tool, and
only the free longitudinal end of the longitudinal first anchoring part is provided with the application tool engagement structure.

21. The human implant arrangement as recited in claim 16, wherein each of the longitudinal first anchoring part, the longitudinal second anchoring part, and the connection part are defined by one single integral plastic body.

22. The human implant arrangement as recited in claim 16, wherein,
the connection part has a longitudinal length,
each of the longitudinal first anchoring part and the longitudinal second anchoring part have a longitudinal length, and
the longitudinal length of the connection part is less than the longitudinal length of each of the longitudinal first anchoring part and the longitudinal second anchoring part.

23. The human implant arrangement as recited in claim 16, wherein,
the connection part has a longitudinal length,
the human implant arrangement has a total longitudinal length, and
the longitudinal length of the connection part is less than ¼ of the total longitudinal length of the human implant arrangement.

24. The human implant arrangement as recited in claim 16, wherein,
the connection part comprises a ring structure which lies in a ring plane,
the lateral self-anchoring elements of each of the longitudinal first anchoring part and the longitudinal second anchoring part lie in a plane, and
the ring plane of the ring structure lies in the plane of the lateral self-anchoring elements of each of the longitudinal first anchoring part and the longitudinal second anchoring part.

25. The human implant arrangement as recited in claim 16, wherein the connection part comprises a length adjusting device which is configured to adjust a longitudinal distance between the longitudinal first anchoring part and the longitudinal second anchoring part.

26. The human implant arrangement as recited in claim 25, wherein the length adjusting device comprises a unidirectional snap-in structure.

27. The human implant arrangement as recited in claim 16, wherein each of the lateral self-anchoring elements of each of the longitudinal first anchoring part and the longitudinal second anchoring part have an anchor angle which is less than 55°.

28. The human implant arrangement as recited in claim 16, wherein,
the longitudinal first anchoring part further comprises a center body which holds the lateral self-anchoring elements of the longitudinal first anchoring part,
the longitudinal second anchoring part further comprises a center body which holds the lateral self-anchoring elements of the longitudinal second anchoring part,
a total lateral extension of the human implant arrangement in a plane of the lateral self-anchoring elements is not larger than 10 mm, and
the total lateral extension is more than 300 % of a lateral thickness of each center body.

29. The human implant arrangement as recited in claim 16, wherein,
the connection part has a lateral extension,
each of the lateral self-anchoring elements of the longitudinal first anchoring part and the longitudinal second anchoring part have a lateral extension, and
the lateral extension of the connection part is not larger than the lateral extension of the self- anchoring elements of the longitudinal first anchoring part and the longitudinal second anchoring part.

30. The human implant arrangement as recited in claim 16, wherein the longitudinal second anchoring part further comprises a holder element which is configured to hold at least one separate anchoring module in place.

31. The human implant arrangement as recited in claim 30, wherein the holder element is a holding stick.

32. The human implant arrangement as recited in claim 16, wherein the lateral self-anchoring elements of the longitudinal second anchoring part are arranged to lie in at least two different self-anchoring element planes.
